(19) United States
(12) Patent Application Publication
(10) Pub. No.: US 2003/0235610 A1
(43) Pub. Date: Dec. 25, 2003

(54) Liposomes containing biologically active compounds

(75) Inventors: Scott A. McLean, Halifax (CA); William R. Campbell, Jamestown, NC (US)

Correspondence Address: FOLEY & LARDNER, P.O. BOX 80278, SAN DIEGO, CA, 92138-0278, US

(73) Assignee: Piedmont Pharmaceuticals, LLC

(21) Appl. No.: 10/366,584

(22) Filed: Feb. 12, 2003

Related U.S. Application Data
(60) Provisional application No. 60/390,508, filed on Jun. 21, 2002.

(51) Int. Cl. A61K 31/522; A61K 31/415; A61K 9/127; A61K 31/192

(52) U.S. Cl. 424/450; 514/263.38; 514/406; 514/569; 514/570

(57) ABSTRACT

The present invention provides compositions of liposomes that contain a biologically active compound and methods of manufacture thereof. Invention methods offer the ability to manufacture liposome compositions containing NSAIDs and other biologically active compounds, and the ability to attain very high encapsulation efficiencies in uniform, stable liposomes. Compositions of liposomes containing anti-viral agents and anti-fungal agents are also provided. Invention liposome compositions are useful for the treatment of a variety of conditions, including, for example, pain and inflammation, bacterial infections and viral infections.

### RELATED APPLICATIONS

This application claims the benefit of U.S. Provisional Application Serial No. 60/390,508, which was filed on Jun. 21, 2002, the contents of which are incorporated by reference herein in their entirety, including all tables, figures, and claims.

### FIELD OF THE INVENTION

The present invention relates to liposome compositions and methods for their manufacture and use.

### BACKGROUND

Liposomes offer a useful mode for administering biologically active compounds. The non-steroidal anti-inflammatory (NSAID) drugs have generally been used for treating pain and inflammation, such as that caused by joint disease, by oral administration. Treatment of inflammation commonly involves oral administration of steroidal or non-steroidal medications and is characterized by redness, swelling, heat, pain, and loss of function. Some non-steroidal anti-inflammatory compounds are available as oral drugs, topical creams, ointments, and gels. But the most effective non-steroidal anti-inflammatory compounds are not available as potent, topically administered compositions. Furthermore, orally administered NSAIDs may cause side effects such as stomach upset, and even congestive heart failure, kidney failure, and toxicity to the lining of the gastrointestinal tract. Existing methods of manufacturing and preparing liposome compositions do not enable the user to prepare a composition containing liposomes encapsulating NSAIDs that can be topically administered with maximum pharmaceutical effect.

### SUMMARY OF THE INVENTION

The present invention provides compositions of liposomes that contain a biologically active compound and methods of their manufacture. The liposomes are preferably multilamellar liposomes suitable for topical administration. In preferred embodiments the liposome compositions utilize a water soluble preservative and a lipid soluble anti-oxidant, and have a high viscosity even though little or no viscosity enhancing agents are present. The methods of manufacture offer the ability to attain very high encapsulation efficiencies, such as encapsulation efficiencies of 50%, or 60% or 70% or 80% or 90% or even 95%. The present methods also offer the ability to manufacture large scale quantities of liposome compositions, at the level of 10 liters or greater.

The liposomes of the present invention contain a biologically active compound and a water soluble preservative. In preferred embodiments, the liposome compositions also contain a lipid soluble anti-oxidant, which is most preferably vitamin E. In other embodiments, at least 75% of the liposomes are from about 1 μm to about 10 μm in diameter, preferably about 3 μm to about 5 μm and up to 1% can be as large as 20 μm in diameter. In still other embodiments, the present compositions contain less than about 1% w/w of a viscosity enhancing agent, and more preferably do not contain any viscosity enhancing agent. Nevertheless, the liposome compositions preferably have a viscosity of at least 20,000 centipoise at 58° C., and more preferably at least about 30,000 centipoise at 58° C. In a preferred embodiment the biologically active compound is a non-steroidal anti-inflammatory compound. In a most preferred embodiment, the non-steroidal anti-inflammatory compound is naproxen, ketoprofen, celecoxib, or indomethacin. Fluconazole and acyclovir, are also preferred biologically active compounds for use in the liposome compositions. But in other embodiments any active compound can be contained in the liposome.

By “liposome” is meant a spherical vesicle bounded by an ordered lipid bilayer and enclosing an aqueous phase. The lipid bilayer of liposomes is usually made of natural or synthetic phospholipids, but can also be made of non-phospholipids. For example, the lipid bilayer can also contain cholesterol and other lipids. Liposomes can contain some portion of solid particle, but are distinguished from microcrystals such as described in U.S. Pat. No. 5,091,188 to Haynes, which comprise a solid crystal particle coated with a lipid layer that is not an ordered bilayer with the heads and tails of the phospholipid lined up, but rather is a disordered coating. In the present application the liposomes have less than 25% of the volume inside the lipid vesicle being solid, i.e., any type of crystalline formation. The lipid bilayer of liposomes is an ordered bilayer, meaning that the molecular “head” and “tail” structures of the lipids are lined up next to one another.

By a compound that is “pharmacologically active” is meant that the compound has some measurable effect when administered to a human or animal body, such as a reduction in pain, inflammation, anti-viral or anti-fungal properties, or another measurable biological property. By a compound that is “biologically active” is meant that the compound has some measurable effect in an in vitro or in vivo assay designed to predict the effect of the compound if administered to a human or animal body. Persons of ordinary skill in the art will realize that there exist numerous assays to test wide varieties of compounds for a particular activity or property. Preferred assays are those that can be demonstrated to be scientifically and statistically meaningful in terms of predicting the activity being tested for, preferably assays that produce a result within three standard deviations. Biologically active compounds may therefore also have an in vivo pharmacological activity. Biologically active compounds suitable for use in the present invention include, but are not limited to, dermatological agents, anti-bacterial agents, anti-viral agents, anti-fungal agents, anti-convulsants, anti-hypertensives, anti-cancer agents, antibiotics, anti-pseboria agents, anti-psoriasis agents, immunomodulators, hormones, proteins, peptides, and the NSAIDs. By a “preservative” is meant an ingredient added to the liposome composition that prevents microbes from substantially growing and multiplying in the formulation. Growth and multiplication of microbes is substantial when it changes the viscosity, stability, or other important property of the composition. Some microbe growth is allowed, as long as it does not negatively impact the physical, chemical, or therapeutic properties of the composition rendering it unsuitable. Microbes can be bacteria, yeast, or molds. By “water soluble” is meant that the ingredient has a solubility in water in excess of 100 μg/ml (or 0.01%) in water. In other embodiments, the ingredient can have a solubility in water in excess of 1 mg/ml (0.1%).

Liposomes of the present invention can be unilamellar (having one lipid bilayer) or more preferably are multilamellar. Liposomes that are “multilamellar” have multiple layers or membranes. This type of liposome has layers of lipid bilayers with an aqueous fluid spaced in between the lipid bilayers. Multilamellar liposomes have at least two layers of lipids. The liposomes of the present invention have widely varying sizes, but preferably are of uniform size in each batch preparation. The liposomes may be up to 20 μm, 25 μm, or even 30 μm. But in preferred embodiments about 95% of the liposomes will be from about 1 μm to about 10 μm in diameter. In one embodiment, the majority of liposomes in a composition manufactured according to the present methods will be from about 3 μm to about 5 μm. The actual diameters of the liposomes will be a function of the cooling curve followed and the length and vigor of stirring or vortex hydration, when those processes are used in the manufacture of the liposomes. In still other embodiments, the liposomes can be multilamellar liposomes where a single larger liposome encapsulates one or more smaller liposomes.

The present invention also provides methods of manufacturing the above-described liposomes. The methods are suitable for manufacturing large quantities of liposome compositions, such as 2,000 liters, 5,000 liters, 10,000 liters, 20,000 liters, and even up to 50,000 liters. The methods involve providing an aqueous solution, and providing a composition of phospholipids containing a biologically active compound at a temperature at least 2° C. greater than the temperature of the aqueous solution. The aqueous and phospholipid compositions are combined to manufacture liposomes encapsulating the biologically active compound. The phospholipid composition is at a temperature at least 2° C. higher than the temperature of the aqueous composition when the compositions are combined. In various embodiments the biologically active compound is encapsulated in the liposomes with an encapsulation efficiency of at least 20%, or at least 50%, or at least 70%, or at least 90%, or even least 95%. In some embodiments, the temperature of the phospholipid composition is about 6° C. or about 10° C. greater than the temperature of the aqueous composition when the two compositions are combined. In the preferred embodiments the temperature of the phospholipid composition is about 58° C. or less and the temperature of the aqueous composition is 8-10° C. lower when the two compositions are combined, e.g., 60° C. and 52° C. or 50° C., or 60° C. and 50° C., or 59° C. and 51° C. or 50° C., or 58° C. and 50° C. or 49° C. or 48° C. All temperatures are ±2° C. The term “about” means ±5%. The phospholipid composition is preferably never subjected to temperatures greater than about 60° C.-65° C., to ensure that none of the ingredients therein are degraded, allowing for optimal liposome formation.

In a most preferred embodiment the temperature of the aqueous composition is about 50° C.±2° C. and the temperature of the phospholipid composition is about 58° C.±2° C. The mixture can be cooled over a period of at least two hours, and preferably is cooled at a rate of about 6° C. per hour. In a preferred embodiment the biologically active compound is a non-steroidal anti-inflammatory, and more preferably is naproxen, ketoprofen, indomethacin, or celecoxib. In other preferred embodiments the biologically active compound is acyclovir or fluconazole. In a most preferred embodiment the mixture is cooled to about 28° C. at a rate of about 6° C. per hour. The non-steroidal anti-inflammatory compounds include, but are not limited to, celecoxib, rofecoxib, naproxen, ketoprofen, diflunisal, fenoprofen, indomethacin, meclofenamin acid, phenylbutazone, piroxicam, salsalate, sulindac, or tolectin. acetyl salicylic acid, ibuprofen, and choline salicylate. But in other embodiments the biologically active compound can be a compound other than an NSAID, such as an anti-viral or anti-fungal compound.

The “encapsulation efficiency” refers to the amount of the active compound that is encapsulated in liposomes by the method relative to the amount of active compound available in the method for encapsulation. The encapsulation efficiency is conveniently expressed by the equation: E_{c}=(total encapsulated drug×100)/total drug, where E_{c} is the encapsulation efficiency. The encapsulation efficiency can be affected by selection of the appropriate types of lipids for the particular application, the shape and size of the vessel in which the procedures are carried out, the amount and size of solid contact masses and degree of vacuum during evaporation and vortex hydration when used in the manufacturing process of prior art liposomes, and the temperature utilized during the manufacturing procedure. An advantage of the present methods is that the number of variables potentially affecting encapsulation efficiency are reduced by eliminating certain requirements, such as the presence of contact masses or vacuum evaporation.

In another aspect the present invention provides methods for decreasing inflammation or pain in a mammal by topically applying to the area where inflammation or pain is to be decreased one of the above-described compositions. In one embodiment the biologically active compound is provided at a concentration in the liposomes of at least about 0.5% w/w. By a “topical” application is meant that the composition is applied to the exterior skin of the treated subject. The skin may be broken or unbroken, and topical application includes application to a lesion on the skin. Topical does not refer to “ocular application,” which is application to the cornea of the eye. “Internal application” includes application to any body cavity, such as the mouth, throat, ear, nose, lung, bronchi, vagina, or rectum. Internal application also refers to compositions that are inhaled (e.g., to treat the lungs or bronchi) or application to an organ in the process of being transplanted.

In another aspect the present invention provides pharmaceutical compositions containing one of the above-described compositions. The invention also provides methods of manufacturing a medicament or pharmaceutical composition by providing a composition of the present invention.

In another aspect the present invention provides methods of treating viral, fungal, and bacterial infections. The methods involve topically, externally, or internally applying a liposome composition of the present invention to the area to be treated. The viral infection can be a herpes infection and the compositions applied to lesions caused by the herpes virus. In other embodiments the infection can be a fungal infection treated by applying a composition to the infected area. In still other embodiments, bacterial infections of the skin, eye, or a body cavity can be treated by topically or externally applying a composition of the present invention.

In another aspect the present invention provides methods of treating a viral infection of the eye. The methods include ocularly applying a liposome composition of the present invention containing an anti-viral agent. In preferred embodiments, the viral infection is herpes and the anti-viral agent is acyclovir.

In another aspect the present invention provides methods of treating a fungal infection. The methods involve topically applying to the infected area a composition of the present invention containing an anti-fungal agent. In preferred embodiments the anti-fungal agent is fluconazole or terbinafine.

The summary of the invention described above is not limiting and other features and advantages of the invention will be apparent from the following detailed description of the preferred embodiments, as well as from the claims.

### DETAILED DESCRIPTION OF THE INVENTION

The present invention provides compositions of liposomes containing biologically active compounds, such as the NSAIDs, anti-virals, and anti-fungals. The inventors discovered unexpectedly that NSAIDs such as naproxen, ketoprofen, indomethacin, and celecoxib and other biologically active compounds such as anti-fungals and anti-virals can be encapsulated into liposomes to result in a composition useful for treating pain and inflammation, preferably at a muscle or skeletal joint. The compositions are also useful for treating viral and fungal infections. But the person of ordinary skill will realize the compositions of the present invention can be used to treat a variety of ailments using various biologically active compounds. For example, when the biologically active compound is an NSAID, the compositions are useful for treating many types of pain, such as a muscle sprain or strain, backaches, toothaches, a high fever, joint pain, or any pain associated with the musculo-skeletal system. The compositions are preferably administered with the liposome composition itself as a carrier, but in various embodiments the liposomes may be administered in a carrier gel or other suitable carrier. The liposomes of the present invention are preferably multilamellar, which are well suited for application to the skin for topical, transdermal delivery of a biologically active compound. The liposomes of present invention allow for slow release, transdermal delivery of a biologically active compound. The liposomes of the present invention can also be made as unilamellar liposomes in those embodiments where injection is desired as a mode of administration. Unilamellar liposomes can be made by filtering the liposomes or by utilizing a french press or other high shear methods of reducing the size of liposomes or screening for unilamellar liposomes.

It has been found that utilizing the compositions and methods of the present invention enables the administration of biologically active compounds at a localized site where the composition is applied. It has been found that the concentration of the biologically active compound at the treated site is about the same using the present methods as that achieved using oral administration of the compound. But the concentration of the biologically active compound systemically is about 1/10^{th} that achieved when the compound is administered orally. Therefore, the present invention allows for the administration of therapeutic levels of biologically active compounds without the undesirable systemic effects that occur with oral administration.

U.S. Pat. Nos. 4,761,288 and 4,897,269, both to Mezei, are both hereby incorporated by reference in their entirety, including all charts and drawings. The liposomes of the present invention do not contain a lipid soluble preservative as found in liposomes of the prior art. Rather, the liposomes of the present invention utilize a water-soluble preservative that can function as an antimicrobial, which is preferably a benzethonium salt, such as benzethonium chloride. But other water soluble preservatives will also find use in the invention, such as benzoic acid, and benzylkonium salts such as benzylkonium chloride. It was discovered unexpectedly that the choice of the preservative is important in order to achieve stable liposomes, as lipid-soluble preservatives can weaken and destabilize the liposomes' structure due to microbial growth, leading to an unstable composition with low viscosity. Other water-soluble preservatives can be used and are advantageously selected to be active at the pH of the composition.

In a preferred embodiment, the present compositions also contain vitamin E as a lipid-soluble anti-oxidant. Anti-oxidants act as free radical scavengers, facilitating the achievement of maximum stability for the liposomes. Methylcellulose or other viscosity enhancing agents are included in liposome compositions that are to be applied to the skin in order to achieve sufficient viscosity and avoid a fluid composition. In a most preferred embodiment, the present compositions include vitamin E as an anti-oxidant and includes less than 2% w/w or less than 1.5% or less than 1.0%, or less than 0.5%, or less than 0.25% of a viscosity enhancing agent. More preferably, the compositions do not include any methylcellulose or any other viscosity enhancing agents. This is most desirable for achieving maximum transdermal penetration of the active compound. In one embodiment at least 50% of the Vitamin E is present in the lipid bilayers of the liposomes. In other embodiments, at least 70% or 80% or 90% or 95% of the vitamin E is present in the lipid layers of the liposomes.

By “viscosity enhancing agents” is meant an agent that is added to the composition to increase the viscosity. A viscosity enhancing agent will increase the viscosity of the composition by at least 10,000 centipoise at 25° C. Viscosity enhancing agents include, but are not limited to, methyl cellulose, alginic acid, gelatin, acacia (gum Arabic) carbomer, and cetostearyl alcohol. Phospholipids are not considered viscosity enhancing agents within this definition. The viscosity enhancing agent will increase the viscosity by at least 10,000 centipoise versus its absence, and in other embodiments can increase the viscosity by 20,000 or 30,000 centipoise (to as high as 40,000 or 50,000 centipoise) versus its absence in the composition. Thus, in various embodiments the liposome compositions of the present invention contain less than 2% w/w or less than 1%, less than 0.5%, or even 0% of viscosity enhancing agents. For example, in various embodiments the compositions contain less than these quantities of organic or inorganic salts, such as salts of hydrochloric acid, nitric acid, sulfuric acid, phosphoric acid, carbonic acid, hydrobromic acid, or hydroiodic acid. The compositions also preferably contain less than 2% w/w or less than 1% or even 0% of potassium bromide, potassium chloride, potassium dihydrogen phosphate, dipotassium hydrogen phosphate, potassium sulfate, potassium iodide, potassium nitrate, lithium bromide, lithium chloride, lithium iodide, lithium nitrate, lithium sulfate, ammonium bromide, ammonium chloride, ammonium carbonate, ammonium hydrogen carbonate, ammonium dihydrogen phosphate, diammonium hydrogen phosphate, ammonium iodide, ammonium nitrate, ammonium sulfate, sodium bromide, sodium carbonate, sodium chloride, sodium hydrogen carbonate, sodium dihydrogen phosphate, disodium hydrogen phosphate, sodium nitrate, sodium phosphate, and sodium sulfate. Other salts that preferably are present in the compositions at less than 2% w/w or less than 1% or even 0% include alkanolamine chloride, sulfate, phosphate, salts of benzoic acid, acetic acid, salicyclic acid, oxalic acid phthalic acid, gluconic acid, 1-naphthalenesulfonic acid, 2-naphthalenesulfonic acid, tartaric acid, maleic acid, malonic acid, succinic acid, fumaric acid, propionic acid, ascorbic acid, mandelic acid, malic acid, citric acid, triethanolammonium chloride, triethanolammonium dihydrogen phosphate, triethanolammonium sulfate, sodium benzoate, potassium benzoate, ammonium benzoate, sodium acetate, potassium acetate, ammonium acetate, sodium salicylate, potassium salicylate, ammonium salicylate, sodium oxalate, potassium oxalate, ammonium oxalate, sodium phthalate, potassium phthalate, ammonium phthalate, sodium gluconate, potassium gluconate, ammonium gluconate, ammonium 1-naphthalenesulfonate, potassium 2-naphthalenesulfonate, ammonium 2-naphthalenesulfonate, sodium 2-naphthalenesulfonate, potassium tartarate, sodium maleate, potassium maleate, sodium malonate, sodium succinate, sodium fumarate, sodium propionate, triethanolammonium propionate, sodium ascorbate, triethanolammonium ascorbate, potassium ascorbate, sodium mandelate, sodium malate, sodium citrate, potassium citrate, and triethanolammonium citrate.

It was discovered unexpectedly that a liposome composition of the present invention results in a stable liposome composition with high viscosity. In various embodiments the compositions have a viscosity of at least 10,000 centipoise, or at least 20,000 centipoise, or at least 30,000 centipoise, or at least 40,000 centipoise, or at least 50,000 centipoise, or at least 60,000 centipoise, or at least 70,000 centipoise, all at 58° C., without the presence of any methyl-cellulose or other viscosity enhancing agents. Because the methylcellulose and other viscosity enhancing agents are not present in the formulations, transdermal penetration is increased substantially. In one embodiment, oleyl alcohol may be added to enhance the transdermal penetration of the biologically active compound that is in the composition but is present outside the liposomes. Without wanting to be bound by any particular theory, it is believed that it is the combination of the water soluble preservative and the lipid soluble anti-oxidant that provides stability to the liposomes. This enables the liposomes to be stable and also have a high viscosity. The high viscosity is possible even though the liposome composition contains very little or no viscosity enhancing agents. It is believed viscosity enhancing agents impair the movement of active compound across the skin. The present compositions offer the superior property of a maximum degree of skin penetration in the administration of the active while retaining a sufficient viscosity. The present compositions are “stable” meaning that they can be stored for at least 6 months, 1 year, or 2 years without changing the chemical or physical properties of the composition. This means that bacterial growth does not occur to an extent that affects the potency, stability, or safety of the compositions. Biological assays of stable compositions will produce the same result within 10% or within 5% of the originally scored value. In various embodiments 50-60% of the liposomes are from about 3 μm to about 5 μm in diameter, with about 20-25% of the remaining liposomes having a diameter of less than 3 μm and the other 20-25% of the liposomes having a diameter of greater than about 5 μm. The term “about” means plus or minus 5%. In other embodiments, at least 95% of the liposomes are less than 10 μm in diameter.

The present invention provides methods of manufacturing liposome compositions of the present invention. The present inventors discovered unexpectedly that very high encapsulation efficiencies can be achieved with the present methods. The present methods involve unique procedures that result in superior encapsulation efficiency and enable the large scale production of commercial quantities of liposome compositions. The methods involve the use of a temperature straddle. A “temperature straddle” means that the aqueous and lipid phases of the starting materials are at different temperatures when combined, with the lipid phase typically being at a higher temperature than the aqueous phase. In preferred embodiments, the lipid phase is maintained at a temperature at least 2° C., and more preferably about 6° C., and most preferably 7°, 8°, 9°, or 10° C. above the temperature of the aqueous phase at the time the two phases are combined. In a most preferred embodiment, the lipid phase is maintained at a temperature of about 58° C.±2° C., and the aqueous phase is maintained at about 50° C.±2° C. In other embodiments, the lipid phase is maintained at a temperature of about 60° C.±2° C., and the aqueous phase is maintained at about 52° C.±2° C.; or the lipid phase can be maintained at any of the temperatures of about 59° C., 57° C., 56° C., 55° C., 54° C., 53° C., or 52±2° C., and the aqueous phase is maintained at any of the temperatures of about 58° C., 57° C., 56° C., 55° C., 54° C., 53° C., 52° C., 51° C., 50° C., 48° C. 47° C., 46° C., or 45° C., all ±2° C., as long as the criteria above are satisfied. The actual temperatures utilized will depend on the specific formulations used. Different lipid formulations can be preferably maintained and combined with the aqueous phase depending on the precise composition of the lipid utilized. If the temperature is too high (e.g., greater than about 65° C.), lipid ingredients may be denatured or otherwise broken down. Thus, it will make it more difficult to form the liposome, and resultant compositions (if any) may be unstable. Therefore, different temperatures may be desirable dependent on the actual lipid composition used.

The method involves rapidly combining the two phases through vortex hydration and temperature shock. This is preferably performed using vessels equipped with an in-line regulating tee and using a dispersing pump to pull the two phases together. When the aqueous and lipid phases are combined, the liposomes are formed. The formation of the liposomes preferably occurs almost instantaneously, e.g., within 10 seconds or 15 seconds or 20 seconds or 30 seconds, depending on the volume of materials used. The mixture can also be passed through a mesh to eliminate clumps of liposomes, thereby enhancing hydration and subsequent formation of liposomes. After thorough hydration of the lipid phase, the composition enters the cooling phase. The cooling process is preferably a slow process, with a maximum cooling rate that is preferably about 6° C. per hour. In other embodiments, the cooling can be at a rate of 4-8° C. per hour, preferably about 6° C. per hour, but can also be allowed to sit and cool naturally over a period of days. In a preferred embodiment the process is complete when a temperature of 28° C. has been reached. In various embodiments it may be desirable to vary the cooling curve, for example, by cooling the composition faster or slower depending on the specific active involved.

The present invention provides methods of treating pain and inflammation by applying the compositions to the skin where pain or inflammation is present or developing. The compositions have been found to be highly effective for reducing the inflammation and easing pain. For example, joint pain is one of the most common minor pain afflictions, affecting millions of people. The present compositions that contain NSAIDs have been found to dramatically reduce joint pain and inflammation when applied to the skin at the joint. In particularly preferred embodiments, the NSAID is ketoprofen or naproxen. The compositions have the advantage of enabling the treated person to receive NSAIDs without the risks associated with oral administration.

The NSAIDs have been shown to have inhibitory effects on prostaglandin and leukotriene synthesis, to have antibradykinin activity, as well as to have lysosomal membrane-stabilizing action. Like several other NSAID compounds, ketoprofen has not previously been successfully encapsulated in liposomes. But using the presently described methods, the inventors have surprisingly been able to produce compositions of liposomes encapsulating naproxen, ketoprofen, indomethacin, and celecoxib useful for treating pain and inflammation. Naproxen is a member of the arylacetic acid group of NSAID drugs, the chemical name being (S)-6-methoxy-alpha-methyl-2 naphthylene acetic acid. The chemical name of ketoprofen is 2-(3-benzoylphenyl) proprionic acid. The ketoprofen liposome composition produced by the present methods is useful for treating various disorders causing pain and inflammation, such as rheumatoid arthritis, osteoarthritis, ankylosing spondodylitis, juvenile arthritis, tendinitis, bursitis, and acute gout.

COX-2 inhibitors block the activity of the enzyme cyclooxygenase 2. This class of drugs includes celecoxib and rofecoxib and has the distinct advantage over older NSAIDs because they act by selectively inhibiting COX-2, the enzyme involved in the pain and inflammation pathway, while not inhibiting COX-1, which is the enzyme involved in protecting the stomach. The present invention provides liposomes containing the COX-2 inhibitors celecoxib, rofecoxib and valdecoxib, for example. These liposomes offer a clear advantage over older COX-2 compositions, since they can be topically applied to the treated, local area and eliminate detrimental effects from oral administration of COX-2 inhibitors. Therefore, the present invention offers the clear advantages of these new drugs but eliminates safety concerns about administration. Utilizing the present invention, one is able to take an NSAID and administer it in a novel and much safer manner than has previously been possible, because the present invention is applied topically. This mode of administration bypasses the gastrointestinal system and localizes the effect of the drug to the site of injury and treatment. This is a distinct advantage of the present invention because the oral administration of COX-2 inhibitors has been associated with irritation of the gastrointestinal tract, heart failure, renal failure, and meningitis.

Other biologically active compounds may also be useful in the present invention. For example, acyclovir, fluconazole or terbinafine may be applied to the present invention to treat viral infections and fungal infections. Thus, the present invention provides methods of treating lesions caused by a herpes infection, such as lesions caused by herpes I, herpes II, or shingles. The present invention can be applied to treat a herpes infection of the skin, where a composition of the invention containing an anti-viral agent is applied to the lesions. The invention may also be applied to treating a viral infection of the eye, where the composition is applied to the cornea of the eye. Similarly, the present invention can be applied to treat a fungal infection by applying a composition of the invention containing an anti-fungal agent (e.g., fluconazole or terbinafine) to the infected area.

Although specific embodiments of NSAIDs and anti-fungal liposome compositions are described herein, the present invention is applicable to any biologically active compounds of interest. Biologically active compounds are preferably lipid soluble for addition to the lipid composition prior to formation of liposomes; although water soluble compounds, or compounds that are soluble in both lipid and aqueous milieu may be used herein. Other useful compounds that may be applied to the present invention include pyrethrins, pyrethroids, carbamates, water-insoluble organo-phosphorus compounds, benzoyl ureas, formamidines, triazines, avermectins, milbemycins, or other standard ectoparasiticides, and derivatives, analogs, and mixtures thereof. Cyhalothrin, cypermethrin, flumethrin, alphamethrin, deltamethrin, and permethrin are particularly preferred pyrethroids. Carbamates such as carbaryl and promacyl are also useful. An effective formamidine is amitraz, and an effective triazine is cryomazine. Diazinon, pirimphos methyl, and pirimphos ethyl are especially effective water-insoluble organo-phosphorus compounds. Water-insoluble endoparasiticides are also effectively administered with the present invention including, for example, the thiazoles and other standard anthelmintics, derivatives, analogs, and mixtures thereof. Effective thiazoles include levamisole, dexamisole and tetramisole.

In some embodiments, the liposome compositions of the present invention contain more than one biologically active compound. There is no theoretical limit to the number of compounds that may be incorporated. For example, two or more ingredients can be encapsulated in the same vesicle, or if the active compounds are incompatible, the compounds can be encapsulated separately and the liposome compositions combined to provide a composition with two or more indications, or that treats a single indication with multiple active compounds. Alternatively, one active ingredient can be encapsulated in the vesicle, and the other dispersed in non-encapsulated form in a surrounding water phase.

In preferred embodiments the lipid used in the present invention is phospholipon 90H, which is obtained and purified from soy lecithin and has the chemical name 1,2-dia-cyl-5N-glycero-3-phosphatidyl choline. It is minimum 90% phophatidyl choline and is fully hydrogenated. But the person of ordinary skill will realize that other lipids may also be used in the present invention. For example, the phosphatidylcholine can be of lower purity, or can contain other lipids or carrier materials such as, for example, propylene glycol/ethanol, medium chain triglycerides, oil/ethanol, phosphatidic acid, cholesterol, and phosphatidylinositol. The phospholipid may be any natural or synthetic phospholipid, for example phosphatidylethanolamine, phosphatidylserine, phosphatidylinositol, phosphatidylglycerol, phosphatidic acid, lysophospholipids, egg or soybean phospholipid or a combination thereof. The phospholipid may be salted or desalted, hydrogenated or partially hydrogenated, natural, synthetic, or semisynthetic. Examples of commercially available phospholipids include but are not limited to egg phospholipids P123 (Pfanstiehl, Waukegen, Ill.), Lipoid E80 (Lipoid, Ludwigshafen, Germany); and the hydrogenated soy phospholipids Phospholipon 80H®, 8G®, 90H® and 100H® (Nattermann, Munich, Germany) and 99% pure soy phosphatidyl choline (Avanti Polar Lipids, Alabaster, Ala.).

Dehydrated alcohol and propylene glycol can be used as co-solvents of the lipid phase, and vitamin E acetate can be included as an anti-oxidant. In various embodiments, other lipids or lipid-like substances are used in the invention, such as ceramides, lecithins, phosphatidyl ethanolamines, phosphatidyl serines, cardiolipins, trilinoleins and like compounds. Nonphospholipids may also be used in the present invention. For example, nonphospholipid materials that may be useful include lipid vesicle forming polyoxyethylene fatty esters, polyoxyethylene fatty acid ethers, diethanolamines, long-chain acyl amides, long-chain acyl amino acid amides, long-chain acyl amides, polyoxyethylene sorbitan oleates, polyoxyethylene glycerol monostearates, glycerol monostearates, and mixtures, analogs, and derivatives thereof. The vesicles may also include a steroid, and a charge producing agent. Preferred steroids include cholesterol, hydrocortisone, and analogs, derivatives, and mixtures thereof. Preferred negative charge producing materials are oleic acid, dicetyl phosphate, palmitic acid, cetyl sulphate, retinoic acid, phosphatidic acid, phosphatidyl serine, and mixtures thereof. In order to provide a net positive charge to the vesicles when desired, long chain amines, e.g., stearyl amines or oleyl amines, long chain pyridinium compounds, e.g., cetyl pyridinium chloride, quaternary ammonium compounds, or mixtures of these can be used. A preferred positive charge producing material is hexadecyl trimethylammonium bromide, a potent disinfectant. The use of this disinfectant as the positive charge producing material within the vesicles provides a secondary advantage as the vesicles deteriorate; they act as a sustained release germicide carriers. But any type of lipid vesicle which could carry sufficient quantities of the aqueous phase could be used.

Alternatively, the preservatives mentioned herein may also act as disinfectants, serving as the sole biologically active compound in a liposome formulation. In such embodiments, the disinfectant (for example a benzethonium salt, such as benzethonium chloride) may be included at about 0.01% to about 1%, more preferably at about 0.1-0.5%, most preferably at about 0.2%, in a lipid disinfectant formulation that would remain on the surface to which it is applied (e.g., skin) for prolonged effectiveness.

### EXAMPLE 1 Exemplary Formulation A

This section provides an example of the starting materials for manufacturing a liposome composition of the present invention containing ketoprofen. The ketoprofen liposome composition contains the following ingredients in the listed percentages w/w.

| | |
|---|---|
| PHOSPHOLIPON ®90H | 10.00 |
| Alcohol, dehydrated, USP | 5.00 or less |
| Propylene Glycol, USP | 5.00 |
| Vitamin E acetate | 1.00 |
| Benzethonium chloride | 0.02 |
| Ketoprofen | 1.00 |
| Purified water | 76.98 |

### EXAMPLE 2 Exemplary Method of Manufacture A

This section provides an example of how to manufacture a liposome composition of the present invention containing ketoprofen.

Aqueous Phase. The process is preferably practiced using two jacketed stainless steel vortex hydration chambers. Into the larger of the two chambers, purified water and benzethonium chloride were combined slowly to avoid the formation of foam or surface bubbles. Heat was applied to obtain 50 C±2° C., the target temperature of the aqueous phase. The chamber was covered to prevent evaporation of water and equipped with a bottom port and valve to regulate flow of material out of the vessel.

Lipid Phase. A second stainless steel jacketed mixing vessel was utilized in close proximity to the first. In this secondary chamber dehydrated alcohol and propylene glycol were first combined slowly to avoid formation of foam or surface bubbles. An overhead mixer was started and heat applied to obtain 58±2° C. with a target of 58° C. When the solution reached the target temperature, ketoprofen was added and fully dissolved. PHOSPHOLIPON® 90H and vitamin E acetate were then added and combined with the lipid phase until dissolved/melted. A cover was used on the chamber to prevent evaporation of alcohol throughout the procedure.

Hydration of the Lipid Phase. Valves were opened on the bottom ports of the chamber, and the flow was regulated from both vessels. The aqueous phase and oil phase flowed and met at an in-line regulating tee, and a dispersing pump pulled the two phases together. The mixture was circulated through a 60 mesh dispersing screen to optimize the hydration of the lipid phase. The mixture was then directed to the top of the chamber and the entire process was circulated through the pump, back into the chamber for 10 minutes.

Cooling Phase. After circulation, the chamber jacket was allowed to cool with continued slow mixing until the temperature of the product was 28° C., completing the process. The combination of materials is preferably fast enough to mix thoroughly without causing formation of surface foam or bubbling. The cooling process is preferably slow, with cooling of about 6° C. per hour most preferable.

### EXAMPLE 3 Use of Exemplary Formulations for Treatment

This example presents actual case histories of the use of various liposome compositions of the present invention for the treatment of various problems. All patients treated themselves with a liposome composition of the present invention, and according to the methods described herein.

Case 1. The patient was a 48 year old male suffering from acute gout in the large left toe with acute episodes occurring for the previous 25 years. The patient had been treated for pain with indomethacin tablets (for 5 days) during this time period. The pills were 75 mg sustained release capsules, twice daily. Using the indomethacin tablets the patient reported the pain dropped from 5 to 0 (on a 1-5 scale with 5 being the highest and 0 indicating no pain) over 3-5 days.

The patient was treated with a liposome composition of the present invention containing indomethacin (1%) at 1 gram formulation on the toe twice daily. The patient reported that pain went from a 5 to a 0 in 3 days.

Case 2. The patient was a 30 year old male suffering from episodes of acute gout in the large right toe for the previous 3 years. During that time the patient was treated with indomethacin tablets (75 mg sustained release capsules, twice daily). The patient was treated with a liposome formulation of the present invention containing 1% indomethacin. The patient used 1 gram of liposome formulation on the toe twice daily for 3 days. The patient reported pain went from a 5 to a 0.

Case 3. The patient was a 49 year old female with episodic pain in both wrists for several years. The patient had not previously used any drug treatments for the pain, except for neutriceuticals (e.g., primrose). The patient used a liposome formulation of the present invention containing 1% naproxen. 1 gram was applied to each wrist daily as needed, usually once per day. Pain went from a 5 to a 0 in 1-2 hours.

Case 4. The patient was a 48 year old male with occasional sore elbow and muscular and skeletal pain attributable to a sports injury. The patient was treated with 1 gram of liposome formulation of the present invention containing 1% naproxen twice daily for 7 days. The patient reported that the pain was alleviated.

Case 5. The patient was a 48 year old male suffering from a recurring skin infection on the face, and was diagnosed by a physician with a yeast infection. The patient had used over the counter anti-fungal creams and SPORANOX® (itraconazole) tablets. The infection cleared occasionally but eventually returned. The patient was treated with a liposome formulation of the invention containing 1% fluconazole, using 0.5 grams once per day for 14 days. The patient reported that the infection cleared and had not returned after 90 days.

Case 6. The patient was a 48 year old female with a lip ulcer believed to be caused by herpes simplex virus. The patient had not been treated for the ulcer and tolerated the ulcer until it disappeared naturally, which was normally about 8-9 days after appearing. The patient was treated with a liposome formulation of the present invention containing 1% acyclovir, using 0.5 grams of formulation twice daily for 3 days. The patient reported the ulcer disappeared in 3 days.

Case 7. The patient was a female with hip pain on the right side and had suffered for less than 2 months. The patient treated herself with a liposome formulation of the present invention containing 1.5% naproxen, applying 1.5 grams of formulation to the area of pain three times daily for 7 days. The patient reported the level of pain went from 4 or 5 to 0 by the end of 7 days.

Case 8. The patient was a 16 year old male with pain in the legs, calf and thighs, which was attributable to musculo-skeletal sports injuries. The patient treated himself with a liposome formulation of the present invention containing 1% naproxen, applying 1 gram of formulation twice daily as needed to the injured area. The patient reported the initial pain at 4 or 5 and at 0 after 1 day of treatment.

Case 9. The patient was a 41 year old male who had suffered a broken left wrist and had had the cast removed immediately prior to treatment. The patient treated himself with a liposome formulation of the present invention containing 1% naproxen, using a total of 2 grams of formulation per day on the top and bottom of his wrist for 14 days. The patient skipped some applications during the 14 days and reported that when the formulation was not used the pain was a 4 or 5. When the formulation was applied the patient reported the pain as a 2 on a 5-scale.

Case 10. The patient was a 53 year old male diagnosed by a physician as suffering from acute gout in his right big toe, right knee, and left elbow for the previous 3 years. The patient had been receiving treatment with indomethacin tablets 2-3 times daily. The patient treated himself with a liposome formulation of the present invention containing 1% indomethacin as an adjunct therapy to the indomethacin tablets. This patient reported on a scale of 1-10, with 10 being the highest pain.

The patient reported the toe pain starts as 10, and drops to 4 with administration of the indomethacin tablet. When topical application of the liposome formulation was added, the pain was further reduced to 2-3.

The patient reported the knee pain starts at 8, and drops to 2 with administration of the indomethacin tablet. The pain then rises to 7-8 after 3-4 hours. With topical application of the liposome formulation the patient reported the pain dropped to 2-3.

The patient reported the elbow pain starts at 4 with swelling. With oral administration of the indomethacin tablet the pain drops to 0, but rises again to 3 after 3-4 hours. When the liposome formulation of the present invention was applied, the pain was reduced to 0.

### EXAMPLE 4 Exemplary Formulation B

| % w/w | Ingredient |
|---|---|
| 9.40 | PHOSPHOLPON ®80H |
| 1.00 | Vitamin E Acetate, USP |
| 5.00 | Propylene Glycol, USP |
| 6.00 | Ethanol, (190 Proof), FCC |
| 0.05 | Glucosamine HCL |
| 0.30 | Methysulfonylmethane (MSM) |
| 0.36 | Pregnenolone |
| 0.05 | S-Adenosylmethionene |
| 0.02 | Benzethonium Chloride, USP |
| 0.10 | TENOX ®4 |
| 77.72 | Water, Distilled |
| 100.00 | |

This section provides an example of how to manufacture a liposome composition of the present invention containing a variety of biologically active ingredients. The particular ingredients herein were chosen to impart maximal therapeutic benefit for pain management in a liposome formulation.

Methysulfonylmethane (MSM) is an organic sulfur compound found naturally in the body, and appears to contribute to the production of collagen.

Pregnenolone is a natural product of cholesterol metabolism that is found in high concentrations in the brain and other tissues of the nervous system. It is in turn a precursor in the formation of a series of steroid hormones.

S-adenosylmethionene (SAM-e) is a natural product of the metabolism of the amino acid methionine. It is known to exhibit beneficial effects to relieve stiffness, pain and swelling, especially in the case of osteoarthritis. A SAM-e deficiency in joint tissues contributes to the loss of the gel-like, resilient nature of cartilage.

Glucosamine is found in relatively high concentration in joints and connective tissues, and is involved in cartilage repair and maintenance. Glucosamine is approved for the treatment of arthritis, and has been shown to ease pain and inflammation, increase range of motion and help repair aging and damaged joints in knees and hips.

Vitamin E functions primarily as an antioxidant in protecting against cell damage, such as from toxic compounds and the body's free-radical metabolites. Because it is lipophilic, it is readily incorporated into the lipid portions of cell membranes and carrier molecules.

TENOX® 4 is a food grade antioxidant/preservative containing both butylated hydroxyanizole (BHA) and butylated hydroxytoluene (BHT).

### EXAMPLE 5 Exemplary Method of Manufacture B

This section provides an example of how to manufacture a liposome composition of the present invention containing ketoprofen.

Aqueous Phase. Distilled water (21,761.60 g) was transferred into a 32.00 liter aluminum container. Slowly the following ingredients were added into the container: benzethonium chloride, USP (5.60 g), glucosamine HCL (14.00 g), and S-adenosylmethionene (14.00 g). Each ingredient was added individually until dissolved; and then the mixture was continually stirred for 10 minutes. The mixture was then warmed to about 52° C.-54° C. with a hot plate. The container was kept closed to prevent evaporation of water.

Lipid Phase. Ethanol (190) (1680.00 g) and propylene glycol, USP (1400.00 g) were added to a 7.5 liter stainless steel container. The mixture was then heated to about 58° C.-60° C. The container was kept closed to prevent evaporation of alcohol. The warmed mixture is then stirred with an overhead mixer and 4″ propeller blade. Pregnenolone (100.80 g) and MSM (84.00 g) were then slowly added. Once these ingredients were completely dissolved, the mixture was then filtered. Vitamin E acetate, USP (280.00 g), PHOSPHOLIPON® 80H (2632.00 g), and TENOX® 4 (28.00 g) were then slowly added. The speed of the mixer was adjusted as necessary to obtain adequate mixing, and maintain the heat of the container between 58° C. and 60° C. until all these ingredients were melted or dissolved, for a time of approximately 10 to 30 minutes.

Hydration of the Lipid Phase. The aqueous phase was stirred using an overhead mixer and 5″ anchor blade. The heat supply was removed to both the aqueous and lipid phases, at which point the lipid phase was quickly added into the vortex of the aqueous phase. The mixer speed was adjusted as necessary to achieve optimal hydration of the lipid phase.

Cooling Phase. An ice bath was used to cool the product. Stirring was continued until the temperature of the final product was between about 30° C.-35° C. The mixer was slowed during the cooling phase as necessary to prevent incorporation of air into the product.

### EXAMPLE 6 Use of Exemplary Formulation B for Treatment

This example presents an actual case history of the use of liposome formulation B of the Example 4 of present invention for the treatment of pain.

The patient was a 24 year old female with torn cartilage in the left knee, presenting with pain in the ankles and knees. Liposome formulation B (approximately 1-2 grams) was applied at each ankle and knee twice daily for one week. The patient reported significantly less pain and more flexibility in movement, and has resumed jogging to some extent without the before-treatment level of pain.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

That which is claimed is:

1. A method of manufacturing liposomes containing a biologically active compound, said method comprising:
providing an aqueous composition,
providing a composition of phospholipids containing a biologically active compound, and
combining the aqueous composition and the phospholipid composition to manufacture liposomes encapsulating the biologically active compound;
wherein the phospholipid composition is at a temperature at least 2° C. greater than the temperature of the aqueous composition when combined.

2. A method according to claim 1, wherein said aqueous composition contains a water soluble preservative and said phospholipid composition contains a lipid soluble anti-oxidant; and wherein said biologically active compound is encapsulated in the liposomes with an encapsulation efficiency of at least 20%.

3. A method according to claim 2, wherein the encapsulation efficiency is at least 50%.

4. A method according to claim 3, wherein the encapsulation efficiency is at least 90%.

5. A method according to claim 2, wherein said biologically active compound is a non-steroidal anti-inflammatory compound.

6. A method according to claim 5, wherein said non-steroidal anti-inflammatory compound is selected from the group consisting of celecoxib, diflunisal, fenoprofen, indomethacin, ketoprofen, meclofenamin acid, naproxen, acyclovir, phenylbutazone, piroxicam, salsalate, sulindac, tolectin, rofecoxib, valdecoxib, and combinations of any two or more thereof.

7. A method according to claim 5, wherein said non-steroidal anti-inflammatory compound is selected from the group consisting of celecoxib, naproxen, indomethacin, ketoprofen, and combinations of any two or more thereof.

8. A method according to claim 2, wherein said biologically active compound is an anti-viral agent or an anti-fungal agent.

9. A method according to claim 8, wherein said anti-viral agent is acyclovir and said anti-fungal agent is fluconazole or terbinafine.

10. A method according to claim 1, wherein the temperature of said phospholipid composition is about 8° C. greater than the temperature of said aqueous composition when said phospholipid and aqueous compositions are combined; and wherein said composition further comprises a water soluble preservative and a lipid soluble anti-oxidant.

11. A method according to claim 10, wherein the temperature of said aqueous composition is about 50° C. and the temperature of said phospholipid composition is about 58° C.

12. A method according to claim 11, further comprising cooling the mixture over a period of at least two hours.

13. A method according to claim 11, wherein the mixture is cooled at a rate of about 6° C. per hour.

14. A method according to claim 11, wherein said biologically active compound is a non-steroidal anti-inflammatory compound.

15. A method according to claim 14, wherein said non-steroidal anti-inflammatory compound is selected from the group consisting of celecoxib, diflunisal, fenoprofen, indomethacin, ketoprofen, meclofenamin acid, naproxen, phenylbutazone, piroxicam, salsalate, sulindac, tolectin, rofecoxib, valdecoxib, and combinations of any two or more thereof.

16. A method according to claim 11, wherein said biologically active compound is a non-steroidal anti-inflammatory; said water soluble preservative is a benzethonium salt; and said lipid soluble anti-oxidant is vitamin E.

17. A method according to claim 11, wherein the non-steroidal anti-inflammatory compound is selected from the group consisting of celecoxib, naproxen, indomethacin, ketoprofen, and combinations of any two or more thereof.

18. A method according to claim 11, wherein said biologically active compound is an anti-viral agent or an anti-fungal agent.

19. A method according to claim 18, wherein said anti-viral agent is acyclovir and said anti-fungal agent is fluconazole or terbinafine.

20. A method according to claim 11, wherein the mixture is cooled to about 28° C.

21. A method according to claim 16, wherein said non-steroidal anti-inflammatory compound is selected from the group consisting of celecoxib, diflunisal, fenoprofen, indomethacin, ketoprofen, meclofenamin acid, naproxen, phenylbutazone, piroxicam, salsalate, sulindac, tolectin, rofecoxib, valdecoxib, and combinations of any two or more thereof.

22. A method according to claim 16, wherein said non-steroidal anti-inflammatory compound is selected from the group consisting of celecoxib, naproxen, indomethacin, ketoprofen, and combinations of any two or more thereof.

23. A composition of liposomes comprising:
a biologically active compound,
a water soluble preservative, and
a lipid soluble anti-oxidant;
wherein at least 75% of the liposomes are from about 1 μm to about 10 μm in diameter; and
wherein the composition has a viscosity of at least 10,000 centipoise and contains less than 2% of a viscosity enhancing agent.

24. A composition according to claim 23, wherein said lipid soluble anti-oxidant is vitamin E; and said biologically active compound is a non-steroidal anti-inflammatory compound.

25. A composition according to claim 24, wherein said non-steroidal anti-inflammatory compound is selected from the group consisting of celecoxib, acetyl salicylic acid, choline salicylate, diflunisal, fenoprofen, motrin, indomethacin, ketoprofen, meclofenamin acid, naproxen, phenylbutazone, piroxicam, salsalate, sulindac, tolectin, rofecoxib, valdecoxib, and combinations of any two or more thereof.

26. A composition according to claim 23, wherein said composition has a viscosity of at least 30,000 centipoise.

27. A composition according to claim 24, wherein said non-steroidal anti-inflammatory compound is selected from the group consisting of celecoxib, naproxen, indomethacin, ketoprofen, and combinations of any two or more thereof; and wherein said water soluble preservative is a benzethonium salt.

28. A composition according to claim 23, wherein said biologically active compound is an anti-viral agent or an anti-fungal agent.

29. A composition according to claim 28, wherein said anti-viral agent is acyclovir and said anti-fungal agent is fluconazole or terbinafine.

30. A method for decreasing pain or inflammation in a mammal, said method comprising:
topically applying to the area where the pain or inflammation is to be decreased a liposome composition, said liposome composition comprising:
a non-steroidal anti-inflammatory compound,
a water soluble preservative, and
a lipid soluble anti-oxidant;
wherein at least 75% of the liposomes are from about 1 μm to about 10 μm in diameter; and
wherein said composition has a viscosity of at least 20,000 centipoise and contains less than 2% w/w of a viscosity enhancing agent.

31. A method according to claim 30, wherein said water soluble preservative is a benzethonium salt and said lipid soluble anti-oxidant is vitamin E; and
wherein said non-steroidal anti-inflammatory compound is selected from the group consisting of celecoxib, acetyl salicylic acid, choline salicylate, diflunisal, fenoprofen, motrin, indomethacin, ketoprofen, meclofenamin acid, naproxen, phenylbutazone, piroxicam, salsalate, sulindac, tolectin, rofecoxib, valdecoxib, and combinations of any two or more thereof.

32. A method according to claim 30, wherein said non-steroidal anti-inflammatory compound is selected from the group consisting of celecoxib, naproxen, indomethacin, ketoprofen, and combinations of any two or more thereof, and
wherein said composition has a viscosity of at least 30,000 centipoise.

33. A method for decreasing pain or inflammation in a mammal, said method comprising:
topically applying to the area where the pain or inflammation is to be decreased a liposome composition, said liposome composition comprising:
a biologically active compound,
a water soluble preservative, and
a lipid soluble anti-oxidant;
wherein at least 75% of the liposomes are from about 1 μm to about 10 μm in diameter; and
wherein said composition has a viscosity of at least 20,000 centipoise and contains less than 2% w/w of a viscosity enhancing agent.

34. A method according to claim 33, wherein said biologically active compound is selected from the group consisting of glucosamine, methysulfonylmethane, pregnenolone, S-adenosylmethionene, and combinations of any two or more thereof.

35. A method according to claim 34, wherein said water soluble preservative is a benzethonium salt, and wherein said lipid soluble antioxidant is vitamin E.

36. A method of treating a viral infection in the eye, said method comprising:
ocularly applying a liposome composition, said liposome composition comprising,
an anti-viral agent,
a water soluble preservative, and
a lipid soluble anti-oxidant;
wherein said composition has a viscosity of at least 20,000 centipoise and contains less than 2% w/w of a viscosity enhancing agent.

37. A method according to claim 36, wherein said viral infection is herpes and said anti-viral agent is acyclovir.

38. A method of treating a fungal infection, said method comprising:
topically applying to the infected area a liposome composition, said liposome composition comprising:
an anti-fungal agent,
a water soluble preservative, and
a lipid soluble anti-oxidant;
wherein the composition has a viscosity of at least 20,000 centipoise and contains less than 2% w/w of a viscosity enhancing agent.

39. A method according to claim 38, wherein said anti-fungal agent is fluconazole or terbinafine.

40. A method of treating an infection, said method comprising:
topically applying to the infected area a liposome composition, said liposome composition comprising:
an anti-viral or anti-fungal agent,
a water soluble preservative, and
a lipid soluble anti-oxidant;
wherein the composition has a viscosity of at least 20,000 centipoise and contains less than 2% w/w of a viscosity enhancing agent.

41. A method according to claim 40, wherein said infection is a viral skin infection and said anti-viral agent is acyclovir.

42. A method according to claim 40, wherein said infection is a fungal skin infection and said anti-fungal agent is fluconazole or terbinafine.
